(19) United States
(12) Patent Application Publication
(10) Pub. No.: US 2012/0128716 A1
(43) Pub. Date: May 24, 2012

(54) Vaccine Compositions

(76) Inventor: Eric Edward Worrall, Lampeter Dyfed (GB)

(21) Appl. No.: 13/126,713

(22) PCT Filed: Nov. 3, 2009
(86) PCT No.: PCT/GB09/51480

§ 371 Date: May 31, 2011

(30) Foreign Application Priority Data
Nov. 7, 2008 (GB) 0920404.2
Jan. 21, 2009 (GB) 0900943.2

(51) Int. Cl. A61K 39/145; A61P 31/16; A61P 37/04

(52) U.S. Cl. 424/209.1

(57) ABSTRACT

Vaccine compositions, useful for eliciting an immune response in subjects which is protective against influenza type A virus, comprise influenza type A virus antigen and a bacterial sialidase. An intranasal vaccine against highly pathogenic subtype H5N1 virus, for use in the treatment of poultry, preferably comprises inactivated H5N1 antigen, sialidase from Clostridium perfringens A strain 107 and chitosan. The use of bacterial sialidase to potentiate influenza virus antigen vaccine is also disclosed.

The present invention relates to vaccine compositions useful for eliciting an immune response in subjects which is protective against influenza type A viruses. The invention further relates to compositions which are useful for the vaccination of poultry and other birds against highly pathogenic avian influenza H5N1.

Influenza is an infectious disease of mammals and birds caused by RNA viruses of the family Orthomyxoviridae. Typically, it is transmitted from infected animals by coughs and sneezes, creating aerosols containing the virus, and from birds through their droppings. The disease can also be transmitted through contact with body fluids of infected subjects and with surfaces which have been contaminated with these.

The term “avian influenza virus” is usually understood to mean an influenza type A virus since wild birds are the natural hosts of influenza A virus. Some type A strains are called low pathogenic since such strains (LPAI viruses) are generally of low virulence although they can serve as progenitors of highly pathogenic strains (HPAI viruses). Highly pathogenic strains of influenza A subtype H5N1 are endemic in birds in south east Asia and are believed to represent a long term pandemic threat. These strains are highly contagious between domestic poultry, such as chickens and turkeys. Outbreaks of the disease in poultry farms, where birds are often intensively reared in close contact with one another, have resulted in bird mortality rates of 100%.

Influenza viruses have two main antigenic glycoproteins at their surfaces: haemagglutinin (HA) and neuraminidase (NA) (also known as sialidase). Primary functions of HA are to bind to the sialic acid receptor sites on the cell membranes of the upper respiratory tract and on the surface of erythrocytes and to facilitate the entry of the viral genome into target cells in the host. NA has functions which aid and promote the release of progeny viruses from infected cells and also plays a role in promoting the entry of the virus into a host cell.

The pharmaceutical industry has produced neuraminidase inhibitors for combating influenza infection. These act by preventing or inhibiting the viral neuraminidase from performing its function. Examples of such neuraminidase inhibitors include Zanamivir and Oseltamivir (“Tamiflu”) which act by blocking the activity of the viral neuraminidase in releasing progeny virus particles from infected cells. Unfortunately, drug therapy, using such compounds, is often ineffective after the disease is clinically recognised since the virus is, by this time, already well established. The use of such compounds prophylactically in the treatment of poultry is usually too expensive for the poultry farmers, particularly those in the areas of the world where H5N1 is endemic.

The aim of the present invention is to provide a cost-effective vaccine which can be used in large scale immunisation programmes for prophylactic treatment against influenza A infection.

Accordingly, the present invention provides a vaccine composition comprising an inactivated influenza type A virus antigen and a bacterial sialidase.

It has surprisingly been found that the incorporation into the vaccine composition of a bacterial sialidase has the effect of increasing the potency of the vaccine. Although we do not wish to be bound by theory, it is possible that the potentiating effect of the bacterial sialidase results from the selective removal of sialic acid from the sialated membrane glycolipids present on the surfaces of the host's epithelia by the sialidase thereby preventing any influenza virus from binding at these sialic acid receptor sites. By blocking the entry of the virus into the vulnerable cells of the host, more time is provided for the host's immune response, elicited by the antigen in the vaccine composition, to act against the attacking influenza virus. In addition, the bacterial sialidase will, itself, have an antigenic effect in the host and, thus, will elicit the production of anti-bacterial sialidase antibodies by the host's immune system which may have an effect against the influenza neuraminidase.

Many different bacterial sialidases have been purified and characterized. The bacterial sialidase used in the vaccine composition of the invention will typically be a sialidase that does not require the presence of any metal ion for activity and may be derived from any suitable bacterial source. Bacterial sialidases typically have molecular weights in the range of 67 kD to 90 kD, depending on the species. Examples of suitable sources of sialidase include Pseudomonas aeruginosa, Clostridium perfringens, Clostridium chauvoei and Clostridium septicum. Preferably, the bacterial sialidase is obtained from Clostridium perfringens and more preferably from Clostridium perfringens type A strain 107, a high sialidase strain. The production/purification of bacterial sialidases, including Clostridium perfringens sialidase, is discussed by James T. Cassidy et al; Purification and Properties of sialidase from Clostridium perfringens; J. Biol. Chem; Vol 240, No. 9, September 1965, p 3501-3506. The preparation of a purified sample of sialidase from Clostridium perfringens A strain 107 is described herein in the following Examples. According to the method used herein, cultured bacterial cells were treated to produce a formalinised Cl perfringens toxoid which was subsequently treated with ammonium sulphate and concentrated by dialysis to produce a purified enzyme. As stated above, the Clostridium perfringens sialidase component used in the production of a preferred embodiment of the invention contains toxoid. It is known that the non-toxoided form of the Clostridium perfringens alpha toxin is haemolytic and necrotic and is the cause of infectious necrotic enteritis in chickens. It is believed that the use of the Clostridium perfringens sialidase in the vaccine composition of the invention has the additional effect of improving resistance, in chickens to which the vaccine composition is administered, to infectious necrotic enteritis.

The inactivated influenza A antigen used in the vaccine composition of the invention will be any antigenic material derived from an inactivated influenza A virus. For instance, it may comprise inactivated whole virus particles. Alternatively, it may comprise disrupted virus (split virus) in which immunogenic protein, for example M2 ion channel protein, or glycoproteins are retained. Purified preparations of influenza A membrane glycoproteins, haemagglutinin (HA) and/or neuraminidase (NA) may be used as the antigenic material in the vaccine composition. A vaccine composition according to the invention may comprise one or more types of these antigenic materials. The influenza type A virus used to prepare the vaccine composition will, of course, depend on the influenza A against which a recipient of the vaccine is to be protected. In view of the threat posed by H5N1, it is preferred that the vaccine composition comprises inactivated antigenic material derived from at least one strain of influenza A subtype H5N1. More preferably, the vaccine composition will comprise inactivated antigenic material from more than one strain of H5N1 in order to elicit broader protection in a subject against different strains of H5N1 in circulation.

The vaccine composition may comprise any one or more suitable carrier, excipient, stabiliser or other additive, as is conventional in the art. Since the main point of entry of the influenza virus into a host is via mucosal surfaces in the upper respiratory tract, it is a preferred embodiment of the present invention that the vaccine composition is adapted for mucosal administration and more preferably intranasal administration. Intranasally administered vaccines are, of course, well known and the common general knowledge of the person skilled in the art teaches how vaccines for intranasal administration may be prepared.

It is also well known in the vaccine art to use an adjuvant in association with an antigenic component of a vaccine. Adjuvants are substances which enhance the immune response to an antigen in a subject. Vaccines adapted for mucosal administration, particularly those adapted for intranasal administration, have been prepared using chitosan, in addition to the antigenic material, to take advantage of its ability to increase transcellular and paracellular transport across mucosal epithelium. Chitosan is the general name given to a class of cationic polysaccharides prepared by the deacetylation of chitin. Chitin is a natural biopolymer which occurs abundantly in the exoskeletons of marine crustaceans. Chitosan is a linear cationic polyelectrolyte which is non-toxic, biodegradable and biocompatible and is a white or off-white amorphous translucent solid which is soluble in dilute organic acids. The chitosan may, advantageously, be used in the form of a non-toxic acid addition salt, e.g. chitosan HCl, which is more soluble than chitosan. Such materials are available from NovaMatrix. According to an especially preferred embodiment, the invention provides a vaccine composition for intranasal administration comprising inactivated influenza A antigen, a bacterial sialidase and chitosan. The chitosan will typically be a deacetylated chitin which is at least 70% deacetylated and preferably at least 80% deacetylated. Chitosan (≧75% deacetylated) is available from Sigma-Aldrich. The chitosan may be used in any amount effective to stimulate the immune response of the intranasally administered influenza A antigen. Typical concentrations of the chitosan used in the vaccine composition of the invention are in the range of from 0.05 to 5%, preferably from 0.1 to 2.0%, more preferably 0.2 to 1.0%, by weight based on the volume of aqueous vaccine composition.

The vaccine compositions of the invention, including those formulated for intranasal administration, can be formulated as liquids or as dry powders according to procedures known in the art. Preferably, the vaccine compositions will be formulated as liquids, especially as aqueous dispersions, suspensions or solutions, for administration as drops or aerosols. The use of the vaccine composition of the invention in an aqueous system is especially preferred since this makes it possible to carry out effective, mass vaccination programmes whereby large numbers of birds may be treated quickly and relatively inexpensively by droplet or aerosol administration. Intranasal application, for example by the application of a single droplet of aqueous vaccine to one nostril of a bird being treated, is easy to carry out in the field by unskilled operators. Furthermore, because of the increased levels of mucosal IgA which result from intranasal vaccination, subsequent protection against the natural route of infection is enabled. When chitosan is used as an adjuvant in an aqueous vaccine composition of the invention, it is beneficial, in order to maintain the solubility of the chitosan in the aqueous medium while ensuring that the antigen component of the vaccine is not adversely affected, that the aqueous vaccine composition has a pH in the range of from about 5.0 to 6.5, preferably about 5.0.

The vaccine of the invention will be administered to a subject in an amount effective to elicit an immune response to influenza A virus. It is within the skill and knowledge of the person skilled in the art to determine suitable and effective doses to be administered to a subject. Although the subject to whom the vaccine is administered may be human, it is a preferred embodiment of the invention that the vaccine is formulated for administration to birds, especially poultry. As shown in the following Examples, successful vaccination of poultry against H5N1 Al virus was achieved using a single dose of aqueous vaccine administered intranasally, as a drop of 0.03 ml to one nostril, containing (per 0.03 ml dose) 100 virus HA units, 122 Cl. perfringens sialidase units suspended in 0.5% w/v chitosan solution.

The incorporation of bacterial sialidase into a vaccine containing inactivated influenza antigen, according to the invention, has the effect of increasing the potency of the vaccine. According to a further aspect, therefore, the invention relates to the use of bacterial sialidase to improve the potency of a vaccine, especially a vaccine administered intranasally, containing inactivated influenza antigen. The invention further relates to a method of improving the potency of such a vaccine by the addition of a bacterial sialidase.

### Experimental Methods

### 1. Preparation of Inactivated Virus Antigen

Highly pathogenic H5N1 homologous strains isolated and identified from infected chickens were propagated in eleven day old incubated specific pathogen free embryonated chicken eggs. Using a working seed virus containing 128 HA units/25 μL the eggs were inoculated, by injection into the allantoic sac of each egg, with 0.2 ml of a 1/1000 dilution in phosphate buffered saline pH 7.4 (PBS)+kanamycin 20 mg/ml.

The eggs were, then, incubated at 37° C. for 72 hours. Embryo death was noted within 25-27 hours.

The dead embryos were chilled at 4° C. and the allantoic fluid harvested and clarified by centrifugation at 3000 rpm for 15 minutes to remove unwanted debris.

After centrifugation, the precipitated matter was discarded and the supernatant containing the virus, on testing, was shown to have haemagglutination activity (by causing the agglutination of chicken red blood cells).

The supernatant was treated to inactivate the virus by the addition of 0.1% formalin followed by incubation at 37° C. for 18 hours. The virus was then split with 0.5% v/v chloroform for 18 hours with constant stirring at 4° C. Residual chloroform was removed under a vacuum of 100 mbar for 2 hours at 28° C. and the sample was retested for haemagglutination activity.

Loss of virulence was demonstrated by inoculating eleven day old incubated embryonated chicken eggs with 0.2 ml of the inactivated virus suspension. All embryos were alive after 5 days further incubation at 37° C.

The virus fluid was clarified by filtration and stored at −20° C.

### 2. Preparation of Bacterial Sialidase

(a) A culture medium was prepared by combining:

| | |
|---|---|
| Oxoid L37 peptone | 2% |
| Lactalbumin Hydrolysate | 1% |
| Yeast extract | 0.5% |
| NaCl | 1.0% |

Water to give a volume of 4.0 litre

The culture ingredients (pH 7.4) were placed in a 10 L Pyrex bottle and autoclaved at 121° C. for 30 minutes. Aqueous glucose (50% w/v sterile aqueous glucose solution) was added to the autoclaved culture ingredients to give a glucose content of 1.0% in the final medium. The medium was then cooled rapidly to 37° C. to maintain reducing conditions.

An inoculum of Clostridium perfringens A strain 107 was prepared by reconstituting freeze-dried seed in Robertson's cooked meat broth. The inoculum was added to 400m1 of the culture medium which was then added back to the bulk culture medium to a total volume of 4.0 L. The pH of the medium was maintained at 7.0 by addition of 5/N NaOH during a 3½ hour growth period and the temperature was maintained at 37° C. After the growth period, the culture was cooled and centrifuged to remove cells and other solid matter. The supernatant was collected and to this was added 0.6% v/v formalin and the formalinised liquor was incubated for 18 hours at 37° C. to form the toxoid.

The titer of the receptor destroying enzyme (RDE), i.e. sialidase, in the Clostridium perfringens toxoid was determined by the following procedure.

Using a 96 well U-bottom microplate, doubling dilutions of the toxoid in pH 5.5 phosphate-buffered saline (PBS) were made in 12 wells leaving 25 μl of each dilution in each of the wells. To each of the dilutions in the wells were added 25 μl of 1% washed chicken red blood cells (RBCs) in pH 7.4 PBS followed by gentle mixing of the contents of each well. The sialidase was allowed to adsorb onto the RBCs for 1½ hours at 28° C. 25 μl of 4HA units of formalin inactivated H5N1 haemagglutinin were added to each well and the contents of each well were left for 3 hours, maintaining the temperature at 28° C. The contents of each of the wells were then studied, noting the highest dilution of the toxoid which inhibits agglutination of the chicken RBCs. The degree of inhibition of agglutination (i.e. the titer of enzyme) was recorded as the reciprocal of the highest dilution of toxoid which inhibited agglutination and this was found to be 1024 RDE units/25 μl (40960 units/ml).

The Clostridium perfingens toxoid, prepared as discussed above, was purified and concentrated according to the following procedure. 50% w/v ammonium sulphate was added to the toxoid. The precipitate produced was discarded and the supernatant was retained for further treatment. To the supernatant was added a further 35% w/v ammonium sulphate (to saturation) and the mixture was allowed to stand for 12 hours. After standing, the mixture was found to contain a brown solid. This solid was collected by skimming it off as it floated to the surface of the liquor. The collected solid was then dissolved in distilled water and was dialysed against distilled water for 8 hours to remove residual ammonium sulphate. The dialysate was then concentrated by further dialysis using polyethylene glycol (PEG) (mol. wt. 20,000), according to known techniques, and then stored at −20° C. Using the method described above for determining the RDE titer, the RDE titer of the purified and concentrated substance prepared as described above was determined.

### 3. Stock Chitosan Solution

85% deacetylated chitosan of crab origin (NovaMatrix) was prepared as a 0.5% w/v solution in 1% v/v pH 5.0 aqueous acetic acid, sodium acetate buffer and sterilised by autoclaving at 121° C. for 20 minutes in a tightly sealed bottle.

### 4. Preparation of Vaccine

To every 100,000 HA units of the inactivated virus prepared above were added 3.0 ml (122880 RDE units) of the Clostridium perfringens sialidase (non-purified, non-concentrated) preparation. The inactivated virus and bacterial sialidase were mixed by stirring at 4° C. for one hour. The mixture was then made up to a total volume of 30 ml by the addition of 0.5% w/v acetic acid/acetate buffered sterile chitosan at pH 5.0 and was further stirred for one hour at 4° C.

The final bulk vaccine was tested for sterility and placed in a sterile 30 ml polypropylene bottle incorporating a dropper nozzle delivering 0.03 ml per drop. Each 0.03 ml dose, thus, consisted of 100 HA units of virus and approx. 122 RDE units of bacterial sialidase suspended in 0.5% w/v sterile chitosan, pH 5.0.

### EXAMPLE 1

### A. IgA Response Using Plain Vaccine Without Bacterial Sialidase

The IgA serological response was studied in a group of chickens, each of which had been intranasally vaccinated by the application of a 30 μl drop of an aqueous vaccine to one nostril. The aqueous vaccine contained (a) homologous H5N1 prepared in embryonated eggs and subsequently inactivated by formalin and (b) commercial chitosan (85% deacetylated). The level of inactivated virus per 30 μl dose was 100 HA units conjugated with 0.4% w/v 85% deacetylated chitosan. The intranasal vaccine used in this Example, thus, did not contain any bacterial sialidase. The pH of the vaccine was 5.0.

A total of 30 layer chickens at 9 weeks old were used in the experiment. The chickens were first vaccinated at 12 days old with the intranasal vaccine. A second vaccination, using the same vaccine, was carried out at 9 weeks of age.

The chickens were divided into 6 groups, each group consisting of 5 chickens per group. Tracheal swabs were taken from the first group before the 9 week stage vaccination. Tracheal swabs from the other groups were taken at different stages following the 9 week stage vaccination, as shown below.

Pre: Tracheal swabs were taken from group I at pre vaccination

1: Tracheal swabs were taken from group II one week post vaccination

2: Tracheal swabs were taken from group III two weeks post vaccination

3: Tracheal swabs were taken from group IV three weeks post vaccination

4: Tracheal swabs were taken from group V four weeks post vaccination

5: Tracheal swabs were taken from group VI five weeks post vaccination

The mucosal IgA response of the intranasally vaccinated chickens was detected using an enzyme-linked immunosorbent assay. The reagents, conditions and apparatus used in the ELISA were as follows:

ELISA
Coating Ag: inactivated Al (H5N1) virus (256 HA), 1:1600
Sample: tracheal swab diluted in 300 μl PBS
Sampling: weekly (pre to 5 weeks post vaccination)
Blocking: TEN (Tris Base, EDTA, NaCl)+0.2% Casein overnight
Conjugate: 1:1500 (goat anti chicken IgA-HRP (Bethyl Lab, Inc)
Substrate: ABTS
ELISA reader: Titertek EX at 415 nm

The results (averaged over 5 samples) according to ELISA, shown as the Optical Density at 405 nm, were as follows:

| | |
|---|---|
| Group 1 (pre-vaccination) | 0.33 |
| Group 2 (1 week post vaccination) | 0.44 |
| Group 3 (2 weeks post vaccination) | 0.31 |
| Group 4 (3 weeks post vaccination) | 0.33 |
| Group 5 (4 weeks post vaccination) | 0.37 |
| Group 6 (5 weeks post vaccination) | 0.4 |

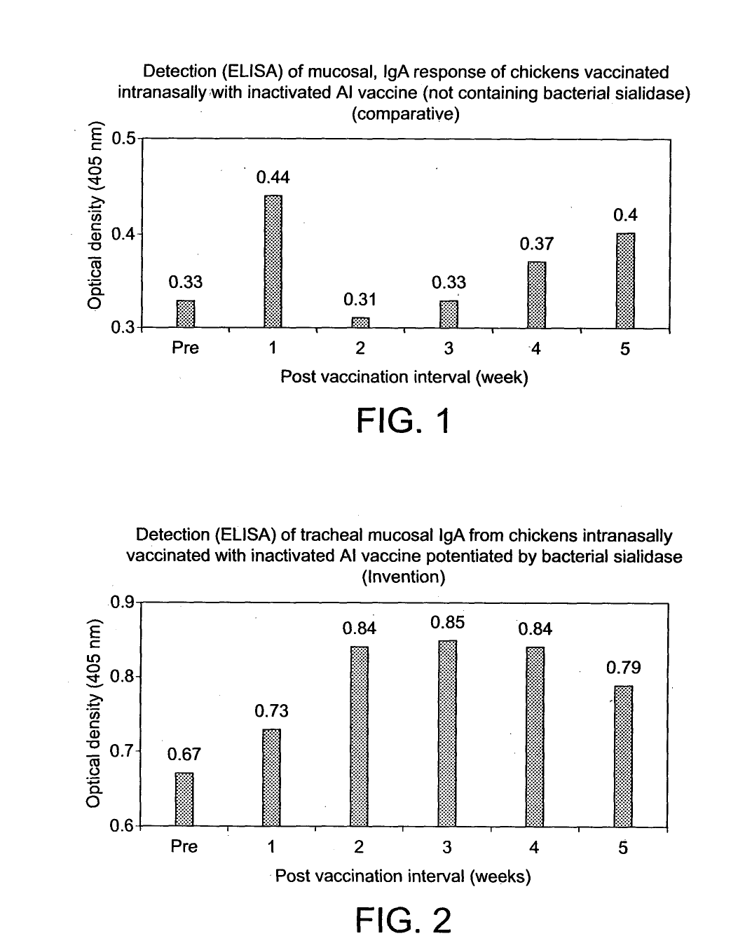

These results are also shown graphically in FIG. 1.

The results demonstrate a mucosal response by the appearance of IgA in the nasal washings taken from the immunised birds which progressively increased over the period from 2 to 5 weeks post vaccination. The early production of IgA within the first week post vaccination might explain the rapid disease control observed in the intervention carried out on diseased flocks. There was a background level of IgA in the non-immunised group, possibly from maternal in ovo antibody.

### EXAMPLE 2

IgA Response in Chickens Intranasally Vaccinated with Vaccine of the Invention

24 chickens (hatched from specific pathogen-free eggs), 20 weeks old, were used in this Example. The chickens were divided into 6 groups each containing 4 chickens. The first group of chickens was not vaccinated. Each chicken in the other groups was intranasally vaccinated by the application of a 30 μl drop of aqueous vaccine to one nostril. The aqueous vaccine contained (a) homologous H5N1 prepared in embryonated eggs and subsequently inactivated by formalin and split with chloroform, (b) commercial chitosan (85% deacetylated) and (c) bacterial sialidase obtained from Clostridium perfringens A strain 107. Component (b) was identical to that used in Example 1. Component (c) (sialidase) was prepared according to the experimental method described above. The intranasal vaccine contained, in each 30 μl dose, 100 virus HA units and 122 perfringens sialidase units suspended in 0.5% w/v chitosan (85% deacetylated), pH 5.0.

Tracheal swabs were taken from the chickens as follows:
Group 1: non-vaccinated
Group 2: 1 week post vaccination
Group 3: 2 weeks post vaccination
Group 4: 3 weeks post vaccination
Group 5: 4 weeks post vaccination
Group 6: 5 weeks post vaccination

The contents of each swab were eluted in 0.4 ml PBS+antibiotic (Kanamycin—10 mg/ml) and stored at 4° C.

The mucosal IgA response of the chickens (non-vaccinated and intranasally vaccinated) was detected by ELISA. The ELISA was performed according to the test procedure as described by Bethyl Laboratories Inc. for the detection of avian IgA. The reagents, conditions and apparatus used in the ELISA were as shown below.

### ELISA Procedure:

Microplate Nunc Maxisorp, U bottom 96 wells

Coating antigen: Split virus: 1/200 of 4HA, diluted in carbonate buffer pH 9.6.

Kept at 4° C. overnight.

Blocking was carried out using 0.2% Casein overnight.

Mucosal samples were diluted 1/5, shaken for 1 hr at room temperature.

Conjugate: affinity purified HRP conjugated anti IgA (Bethyl Lab) 1/2000, shaken for 1 hr at room temperature.

Substrate: ABTS

Read at 415 nm.

The results (mean) according to ELISA, shown as the optical density recorded at 405 nm, were as follows:

| | |
|---|---|
| Control conjugate OD (no samples added) | 0.065 |
| Group 1 (non-vaccinated) | 0.674 |
| Group 2 (1 week post vaccination) | 0.7325 |
| Group 3 (2 weeks post vaccination) | 0.8375 |
| Group 4 (3 weeks post vaccination) | 0.851 |
| Group 5 (4 weeks post vaccination) | 0.8385 |
| Group 6 (5 weeks post vaccination) | 0.7910 |

These results are shown graphically in FIG. 2.

The immune response to a single 30 μl dose of intranasal vaccine containing bacterial sialidase in the tested chickens produced higher levels of IgA than was observed in the chickens given double dose plain vaccine in Example 1, which was without split virus and without bacterial sialidase. The immune response indicated by the raised levels of mucosal IgA, as measured by ELISA, shows that the vaccine containing bacterial sialidase, applied intranasally to immunologically näve chickens, provides a level of protection greater than that achieved using intranasal vaccine containing no bacterial sialidase.

### EXAMPLE 3

Visits were made to commercial chicken breeding farms which had reported and identified HP H5N1 outbreaks of avian influenza. In all cases, mortality had reached alarming levels. The disease was characterised by rapid spread which, from previous experience, would lead to 100% mortality.

Each of the birds in every flock was vaccinated intranasally by the application of a 30 μl drop of the vaccine of the invention prepared as described above to one nostril. Each 30 μl dose contained 100 HA units of inactivated, split virus and 122 RDE units of the bacterial sialidase suspended in 0.5% w/v chitosan, pH 5.0.

### 1. Farm A:

Age of birds: 20 days

Al vaccination: NiI

Mortality: approx. 500 per day

Total population: 25,000

Total losses at intervention: approx. 5000

Intranasal vaccine of the present invention applied

Mortality ceased within 3 days

### 2. Farm B:

Age of birds >5 months

Vaccination history: Intramuscular (IM) vaccine H5N1 RE 1 (commercial vaccine) at five weeks, repeated at 15 weeks

Mortality: approx. 500 birds/day

At the outbreak: intranasal vaccine of the invention administered plus IM H5N1 RE 1 (commercial vaccine)

Mortality continued, 20% survived

### 3. Farm C1:

Age of birds: 6 months

Vaccination history: H5N2 (commercial vaccine), administered intramuscularly, at 6 weeks, repeated at 16 weeks

Mortality: approx. 1000/day

At outbreak: intranasal vaccine of the invention administered plus IM H5N2 (commercial vaccine)

Mortality continued, no survivors

### 4. Farm C2:

Age of birds <4 months

Vaccination history: IM vaccine H5N2 (commercial vaccine) at 5 weeks

At outbreak: only intranasal vaccine of the invention applied

Mortality ceased within 5 days

Discussion:

We have shown that during existing avian influenza outbreaks intervention with the vaccine of the invention applied intranasally can dramatically influence the course of the disease in poultry of all ages housed in intensive breeding systems. We further demonstrate from the above interventions that the use of the intranasal vaccine effectively controls the disease within 2-5 days.
In cases where both the intranasal vaccine of the invention and commercial intramuscular and subcutaneous vaccines were applied simultaneously, control failed and the disease continued.
In cases where the application of intranasal vaccine alone was carried out, complete control was observed and fatalities ceased within 2-5 days.

Conclusion:

High pathogenic avian influenza disease in poultry is peracute. In an infected flock, there is seen a dramatic progressive mortality. In such outbreaks, a proportion of the birds are sub-clinically infected although they show no apparent clinical disease.

Reference is made to Farms B and C1 above. Here the farmers had reservations about the effectiveness of the intranasal vaccine of the invention used on its own and decided to vaccinate all birds at the same time with a commercial intramuscular or subcutaneous vaccine with the result that this operation transmitted the virus from sub-clinically infected birds throughout the whole flock. The intranasal vaccine of the invention acts principally as a barrier producing high levels of IgA preventing entry of virus by natural infection routes. Therefore, it was unable, in the short time, to have any effect on a virus mechanically introduced by the needle of a multi-dose automatic syringe. However, when used as a single intervention in Farms A and C2 there was no incidental transmission of live virus by the use of a multi-dose automatic syringe and further natural transmission was controlled within 2-5 days and fatalities ceased completely.

The above field experience clearly demonstrates the effectiveness of the intranasal vaccine of the invention as a first line of defence preventing access of live virus to susceptible birds by the stimulation of the common mucosal immune system, blocking virus entry and eliciting production of mucosal IgA both before and during an outbreak.

### EXAMPLE 4

In a flock of 6,700 laying chickens (breed Hi sex), which formed part of a total flock of 60,000 birds, abnormal mortality started when 17 chickens were found dead. All of the chickens in the flock, at the point when this abnormal mortality was observed, were 28 weeks old. The cause of death of the 17 chickens was identified as HP H5N1 infection, confirmed by rapid diagnostic testing. It was observed that egg production by the chickens in the flock started to drop subsequently as mortality in the flock increased due to the spread of the infection. On the sixth day after the day on which abnormal mortality was first observed, 611 chickens died. On that day, all of the surviving chickens in the flock were vaccinated using the vaccine prepared as described above in the Experimental method. The vaccine was administered intranasally to each of the chickens by the application of a 30 μl drop to one nostril. Each 30 μl dose of the intranasal vaccine contained 100 virus HA units and 122 sialidase units from Clostridium perfringens A strain 107 suspended in 0.5% w/v aqueous chitosan (85% deacetylated), pH 5.0. Subsequent to vaccination, mortality began to decrease and on the fifth day after vaccination (11^{th} day after abnormal mortality was first observed) only three chickens died as a result of the infection. An increase in egg production was subsequently observed. The haemagglutination inhibition (HI) titer of serum increased to a peak 20 days after administration of the vaccine to the chickens.

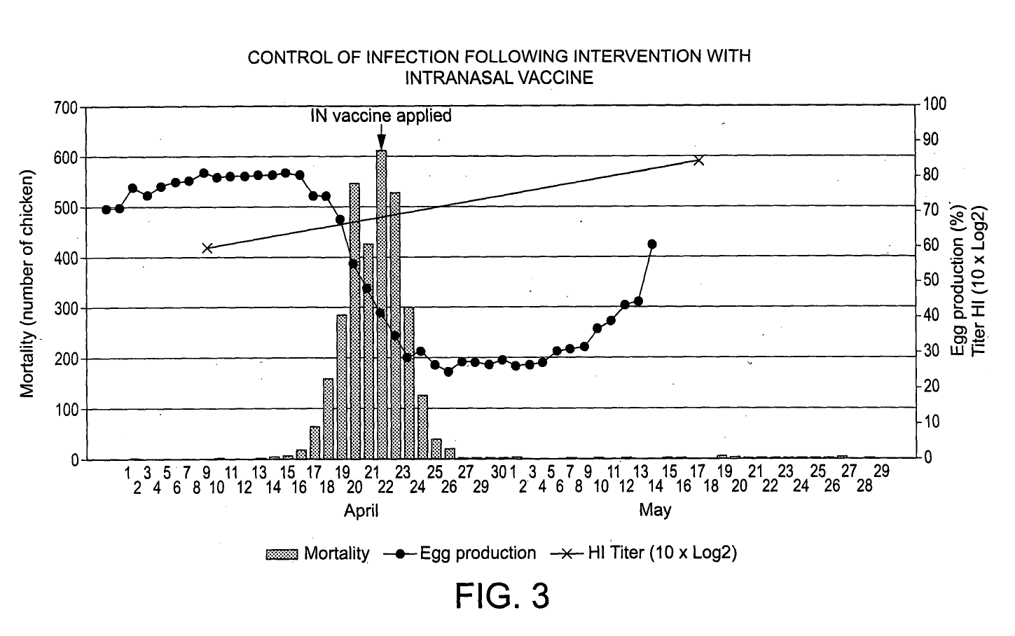

To prevent the spread of the disease to the main flock of birds (original total of 60,000), all of the remaining birds in the main flock were vaccinated with the intranasal vaccine (vaccine composition and dose as above). No further deaths from the H5N1 infection were observed. FIG. 3 shows, separately, mortality of chickens, egg production and haemagglutination inhibition titer over time.

The results displayed in FIG. 3 can be explained in part by the early production of IgA following vaccination. We believe, however, that the response to vaccination also indicates that the bacterial sialidase has an important effect. It is likely that this effect results from the action of the bacterial sialidase functioning in a classical catalyst enzyme and substrate mode on the sialated cell receptors on the mucosal epithelium in the vaccinated birds so as to render these receptors unrecognisable by incoming virus, thus preventing virus attachment and subsequent endocytosis. It has been reported by A. Gottschalk and P. E. Lind, British Journal of Experimental Pathology, Vol. XXX, No. 2, April 1949, and G. K. Hirst, J. Exp. Med., 1942, Aug. 1; 76(2), 195-209, that influenza adsorbed to chicken RBCs elutes spontaneously after a period of time leaving the virus functionally intact but the RBCs irreversibly changed and resistant to further adsorption. The same action could occur with the sialidase where, on completion of the enzyme/substrate, the sialidase is released to repeat its action. Such an action would explain the gradual reduction in mortality which is observed over the period of 2 to 5 days following vaccination. Some birds, at the time of vaccination, were already infected sub-clinically and, thus, the vaccination was not able to prevent their deaths.

Faecal swabs taken monthly from the vaccinated birds, examined by egg inoculation and PCR, failed to isolate or reveal Al virus, thus indicating post-vaccination systemic clearance of virus.

### EXAMPLE 5

Serological responses of chickens vaccinated at 12 days of age were investigated. In all of the following investigations, vaccination was carried out using the vaccine composition prepared as described above in the Experimental method. To each chicken (12 days old) was administered, intranasally, one 0.03 ml dose of aqueous vaccine composition containing 100 virus HA units and 122 sialidase units from Clostridium perfringens A strain 107 suspended in 0.5% w/v chitosan (85% deacetylated), pH 5.0. Vaccination was carried out by the application of 0.03m1 drop of the vaccine composition to one nostril of each bird.

### Investigation No. 1

Thirteen broiler chickens were vaccinated at 12 days of age and the HI Al titer was determined from serum samples taken from the chickens at 32 days of age. The results are shown below in Table 1.

TABLE 1

| No. of chickens | HI titer (log 2) |
|---|---|
| 2 | 0 |
| 2 | 2 |
| 2 | 3 |
| 6 | 5 |
| 1 | 6 |

mean value for HI AI titer (log 2) is 3.54
Coefficient of Variation (CV) is 57.24%

### Investigation No. 2

Six layer chickens were vaccinated at 12 days of age and the HI Al titer was determined from serum samples taken from the chickens at 28 days of age. The results are shown below in Table 2.

TABLE 2

| No. of chickens | HI titer (log 2) |
|---|---|
| 1 | 0 |
| 1 | 3 |
| 4 | 4 |

mean value for HI AI titer (log 2) is 3.17
% CV is 50.59

### Investigation No. 3

Thirty layer chickens were vaccinated at 12 days of age and the HI Al titer was determined from serum samples taken from the chickens at 21 days of age. The results are shown in Table 3 below.

TABLE 3

| No. of chickens | HI titer (log 2) |
|---|---|
| 14 | 0 |
| 1 | 2 |
| 1 | 3 |
| 8 | 4 |
| 2 | 5 |
| 4 | 6 |

mean value for HI AI titer (log 2) is 2.37
% CV is 101.39

The results shown above in Tables 1, 2 and 3 indicate the humoral IgG antibody response as measured by the haemagglutination inhibition test. Although the main activity of the vaccine is the rapid production of secretory IgA and the stimulation of the common mucosal immune system (CMIS), investigations reported above show that humoral antibody is also produced.

The antibody titers of the sero converted birds are within the accepted limits for what is considered to be a protective level of antibody by the O.I.E. (World Organisation for Animal Health) (i.e. 2 log 2).

Further evidence of immunological memory was observed where birds vaccinated at 12 days of age that were accidentally exposed to natural infection by HPAI survived.

Field experience confirms that the control of infections in 2 to 5 days (Examples 3 and 4 and FIG. 3) is a common feature of the action of the vaccine composition of the invention. The production of secretory IgA contributes to an effective blocking action and, as demonstrated in Example 5, systemic production of IgG also occurs. However, the 2 to 5 day control cannot be explained completely by these actions, especially since only a low systemic production of IgG, as illustrated in Tables 1, 2 and 3 in Example 5, will be insufficient to have a direct effect on the cessation of infection in exposed birds. Additionally, a low production of IgG assumed to have occurred in the birds in farms B and C1 in Example 3, which were already infected either naturally or accidentally, was unable to control the disease.

We believe that, in addition to the occupation of receptor sites on the mucosal epithelium by sialidase, there must be recognition of these degraded sites by the lymphocytes, and specifically the cytotoxic T-lymphocytes, which results in the establishment of the cell mediated immune system being a major factor in the control of budding viruses, such as the orthomyxovirus HP H5N1.

It has been reported that in 12 day old birds vaccinated using the intranasal vaccine of the invention, it takes at least 10 weeks post vaccination to reach high circulating IgG HI antibody levels (>64 HI units). This is desirable for the neutralisation of virus which has evaded the first line of the mucosal defences but is of little use at the site of first contact. Cytotoxic T-cells are less strain specific and show broader cross reaction which could be of benefit to neutralise commonly encountered sub-typical mutants.

Evidence of this protective cell mediated immunity was displayed in a farm which had used two intranasal applications of the vaccine of the invention to poultry on the farm when exposed to infection from an adjacent farm separated by only 20 m. No deaths were seen in the intranasally vaccinated poultry on the farm whereas the adjacent farm suffered massive losses of birds. Field experience confirms that contagious infection so close to another farm would be 100% certain to reach the adjacent premises.

1. A vaccine composition comprising influenza type A virus antigen and a bacterial sialidase.

2. A composition according to claim 1, wherein the bacterial sialidase is selected from sialidase of Pseudomonas aeruginosa, Clostridium perfringens, Clostridium chauvoei or Clostridium septicum.

3. A composition according to claim 2, wherein the bacterial sialidase is Clostridium perfringens type A sialidase.

4. A composition according to claim 3, wherein the bacterial sialidase is Clostridium perfringens type A strain 107 sialidase.

5. A composition according to claim 1, wherein the influenza type A virus antigen comprises inactivated whole virus.

6. A composition according to claim 1, wherein the influenza type A virus antigen comprises disrupted virus.

7. A composition according to claim 1, wherein the influenza type A virus antigen comprises purified membrane glycoprotein.

8. A composition according to claim 1, wherein the influenza type A virus is influenza type A subtype H5N1.

9. A composition according to claim 1, which additionally comprises chitosan.

10. A composition according to claim 1 in the form of an aqueous dispersion or solution.

11. A composition according to claim 1 which is adapted for mucosal administration.

12. A composition according to claim 11, wherein the mucosal administration is intranasal administration.

13. A method of eliciting an immune response to influenza type A in a subject which method comprises administering to the subject a composition comprising influenza type A virus antigen and a bacterial sialidase.

14. A method according to claim 13, wherein the subject is a bird.

15. A method according to claim 13, wherein the influenza type A virus is influenza type A subtype H5N1.

16. A method according to claim 13, wherein the bacterial sialidase is Clostridium perfringens sialidase.

17. A method according to claim 16, wherein the Clostridium perfringens is type A 107.

18. A method according to claim 13, wherein the composition additionally comprises chitosan.

19. A method according to claim 13, wherein the composition is adapted for intranasal administration and is administered intranasally to the subject.

20. A method for performing vaccination in poultry comprising administering a composition according to claim 12 intranasally to the poultry.

21. Use of a bacterial sialidase to increase the potency of a vaccine composition comprising influenza A virus antigen.

22. Use according to claim 21, wherein the vaccine composition additionally comprises chitosan.

23. Use according to claim 21, wherein the vaccine is adapted for intranasal administration.

24. Use according to claim 21, wherein the bacterial sialidase is sialidase from Clostridium perfringens type A.

25. Use according to claim 21, wherein the influenza A virus antigen is antigen from highly pathogenic subtype H5N1 virus.
